(19) United States
(12) Patent Application Publication
(10) Pub. No.: US 2024/0144471 A1
(43) Pub. Date: May 2, 2024

(54) METHODS AND DEVICES OF PROCESSING LOW-DOSE COMPUTED TOMOGRAPHY IMAGES

(71) Applicant: TAIPEI MEDICAL UNIVERSITY, Taipei City (TW)

(72) Inventors: Cheng-Yu CHEN, TAIPEI CITY (TW); David Carroll CHEN, TAIPEI CITY (TW)

(21) Appl. No.: 17/978,226

(22) Filed: Nov. 1, 2022

(51) Int. Cl. G06T 7/00; A61B 6/00; G06T 7/12; G06T 7/40; G06T 7/60; G06T 7/70; G16H 30/20; G16H 50/20

(52) U.S. Cl. CPC G06T 7/0012; A61B 6/503; A61B 6/504; G06T 7/12; G06T 7/40; G06T 7/60; G06T 7/70; G16H 30/20; G16H 50/20; A61B 6/032; G06T 2207/10081; G06T 2207/20021; G06T 2207/20081; G06T 2207/30048; G06T 2207/30064; G06T 2207/30101

(57) ABSTRACT

Disclosed are methods and devices of processing a low-dose computed tomography (CT) image. The present disclosure provides a method of processing a low-dose CT image. The method comprises: receiving a first chest image; receiving a first chest image; detecting at least one lung nodule in the first chest image; determining at least one lung nodule region of the first chest image based on the at least one lung nodule; and classifying the at least one lung nodule region based on a first set of radiomics features of the at least one lung nodule region of the first chest image to obtain a nodule score of the at least one lung nodule in the lung nodule region. The first chest image generated by a low-dose CT method.

### FIELD OF THE INVENTION

The present disclosure relates to a method of processing low-dose computed tomography (CT) images and to related devices. In particular, the present disclosure relates to methods of processing low-dose CT images to determine a characteristic of an organ, and to related devices.

### BACKGROUND

Medical advances and declining birthrates have accelerated aging of society, increasing the importance of maintaining health. Thus, regular health examinations are critical to detect possible health problems at the earliest possible stage. Unfortunately, some forms of examination may carry undesirable side effects (e.g., radiation). Therefore, improved methods of health examination with reduced radiation effect are desirable.

### SUMMARY OF THE INVENTION

The present disclosure provides a method of processing a low-dose computed tomography (CT) image. The method includes receiving a first chest image; detecting at least one lung nodule in the first chest image; determining at least one lung nodule region of the first chest image based on the at least one lung nodule; and classifying the at least one lung nodule region based on a first set of radiomics features of the at least one lung nodule region of the first chest image to obtain a nodule score of the at least one lung nodule in the lung nodule region. The first chest image is generated by a low-dose CT method.

The present disclosure provides a method of processing a low-dose computed tomography (CT) image. The method includes receiving a first chest image; extracting a heart region in the first chest image by using a U-Net model; determining a coronary artery calcification (CAC) score of the heart region by an transferred Efficient Net model. The first chest image is generated by a low-dose CT method.

According to another embodiment, the present disclosure provides device of processing a low-dose computed tomography (CT) image. The device includes a processor and a memory coupled with the processor. The processor executes computer-readable instructions stored in the memory to perform operations. The operations include receiving a first chest image; detecting at least one lung nodule in the first chest image; determining at least one lung nodule region of the first chest image based on the at least one lung nodule; and classifying the at least one lung nodule region based on a first set of radiomics features of the at least one lung nodule region of the first chest image to obtain a nodule score of the at least one lung nodule in the lung nodule region. The first chest image is generated by a low-dose CT method.

According to another embodiment, the present disclosure provides device of processing a low-dose computed tomography (CT) image. The device includes a processor and a memory coupled with the processor. The processor executes computer-readable instructions stored in the memory to perform operations. The operations include receiving a first chest image; extracting a heart region in the first chest image by using a U-Net model; determining a coronary artery calcification (CAC) score of the heart region by an transferred Efficient Net model. The first chest image is generated by a low-dose CT method.

### BRIEF DESCRIPTION OF THE DRAWINGS

In order to describe the manner in which advantages and features of the present disclosure can be obtained, a description of the present disclosure is rendered by reference to specific embodiments thereof, which are illustrated in the appended drawings. These drawings depict only example embodiments of the present disclosure and are not therefore to be considered limiting its scope.

FIG. 1 is a diagram of a low-dose computed tomography (LDCT) image processing architecture, in accordance with some embodiments of the present disclosure.

FIG. 2 is a flowchart showing a method of processing a low-dose CT image, in accordance with some embodiments.

FIG. 3 is a flowchart showing a method of processing a low-dose CT image to determine a nodule score of lung nodules, in accordance with some embodiments.

FIG. 4 is a diagram of an image processing architecture, in accordance with some embodiments.

FIG. 5 is a diagram of performance of lung nodule detection, in accordance with some embodiments.

FIG. 6 is a diagram of an image processing architecture, in accordance with some embodiments.

FIG. 7 is a diagram of a classification framework of features of an image, in accordance with some embodiments.

FIGS. 8 and 9 are diagrams of performance, in accordance with some embodiments.

FIG. 10 is a diagram of a nodule score classification procedure of features of an image, in accordance with some embodiments.

FIGS. 11A and 11B show lung images, in accordance with some embodiments.

FIG. 12 is a flowchart showing a method of processing a low-dose CT image to determine a coronary artery calcification (CAC) score, in accordance with some embodiments.

FIG. 13 is a diagram of a CAC determination procedure, in accordance with some embodiments.

FIGS. 14 and 15 are diagrams of performance, in accordance with some embodiments.

FIG. 16 illustrates a schematic diagram showing a computer device according to some embodiments of the present disclosure.

### DETAILED DESCRIPTION

The following disclosure provides many different embodiments, or examples, for implementing different features of the provided subject matter. Specific examples of operations, components, and arrangements are described below to simplify the present disclosure. These are, of course, merely examples and are not intended to be limiting. For example, a first operation performed before or after a second operation in the description may include embodiments in which the first and second operations are performed together, and may also include embodiments in which additional operations may be performed between the first and second operations. For example, the formation of a first feature over, on or in a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Time relative terms, such as “prior to,” “before,” “posterior to,” “after” and the like, may be used herein for ease of description to describe the relationship of one operation or feature to another operation(s) or feature(s) as illustrated in the figures. The time relative terms are intended to encompass different sequences of the operations depicted in the figures. Further, spatially relative terms, such as “beneath,” “below,” “lower,” “above,” “upper” and the like, may be used herein for ease of description to describe the relationship of one element or feature to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly. Relative terms for connections, such as “connect,” “connected,” “connection,” “couple,” “coupled,” “in communication,” and the like, may be used herein for ease of description to describe an operational connection, coupling, or linking one between two elements or features. The relative terms for connections are intended to encompass different connections, coupling, or linking of the devices or components. The devices or components may be directly or indirectly connected, coupled, or linked to one another through, for example, another set of components. The devices or components may be wired and/or wirelessly connected, coupled, or linked with each other.

As used herein, the singular terms “a,” “an,” and “the” may include plural referents unless the context clearly indicates otherwise. For example, reference to a device may include multiple devices unless the context clearly indicates otherwise. The terms “comprising” and “including” may indicate the existences of the described features, integers, steps, operations, elements, and/or components, but may not exclude the existences of combinations of one or more of the features, integers, steps, operations, elements, and/or components. The term “and/or” may include any or all combinations of one or more listed items.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

The nature and use of the embodiments are discussed in detail as follows. It should be appreciated, however, that the present disclosure provides many applicable inventive concepts that can be embodied in a wide variety of specific contexts. The specific embodiments discussed are merely illustrative of specific ways to embody and use the disclosure, without limiting the scope thereof.

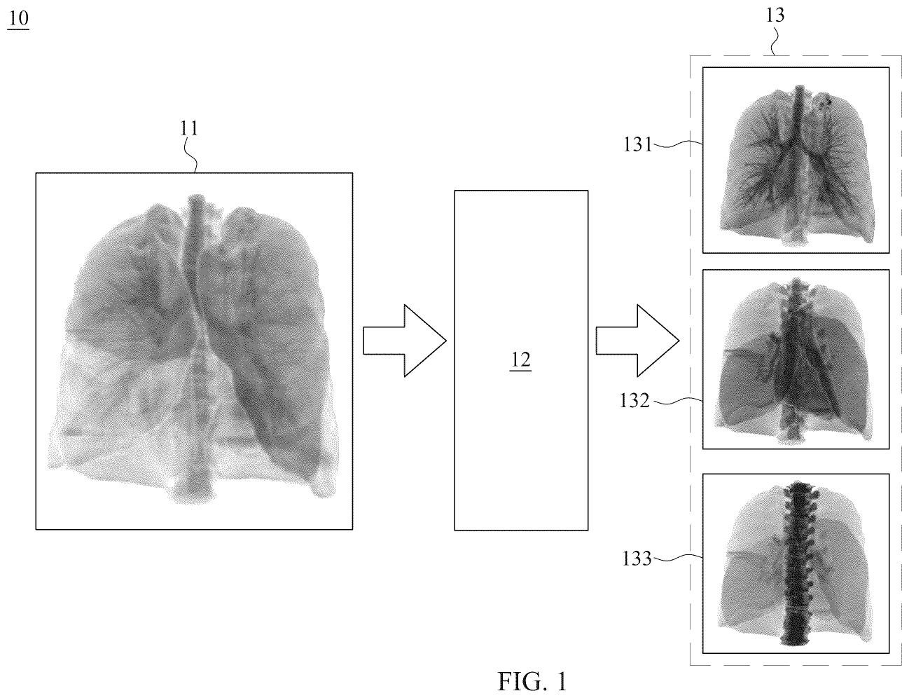

FIG. 1 is a diagram of a CT image processing architecture 10, in accordance with some embodiments of the present disclosure. The CT image processing architecture 10 includes a CT image 11, an image process model 12, and processed images 13.

The CT image 11 can be a full-dose CT image or a low-dose image. The low-dose CT method can be less physically harmful. In some embodiments, the CT image 11 can be a chest CT image of a human. The CT image 11 can include one or more organs of a human. For example, the CT image 11 can include lungs or heart, or bones, such as thoracic vertebrae, ribs, sternum, and/or clavicle. In some embodiments, the CT image 11 can be a two-dimensional (2D) image. In other embodiments, the CT image 11 can be a three-dimensional (3D) image.

The CT image 11 can be input to the image process model 12. In some embodiments, the image process model 12 can include one or more models therein. For example, the image process model 12 can include, but is not limited to, object detection, semantic segmentation, classification, and localization models. In some embodiments, the image process model 12 can analyze pixels in the CT image 11. The image process model 12 can detect each element in the CT image 11. In some embodiments, the image process model 12 can analyze different organs in the CT image 11.

The image process model 12 can output the processed images 13. The processed images 13 can include marks thereon. The processed images 13 can be processed voxel-by-voxel. The processed images 13 can be processed according to different models. In some embodiments, the processed images 13 can be identified as organs. The processed images can include three images 131, 132, and 133, with, for example, lungs identified in image 131, heart in image 132, and thoracic vertebrae in image 133. In some embodiments, the processed images 131, 132, and 133 can show the result of the analysis on different organs. Characteristics of the organs can be analyzed to ascertain the condition thereof.

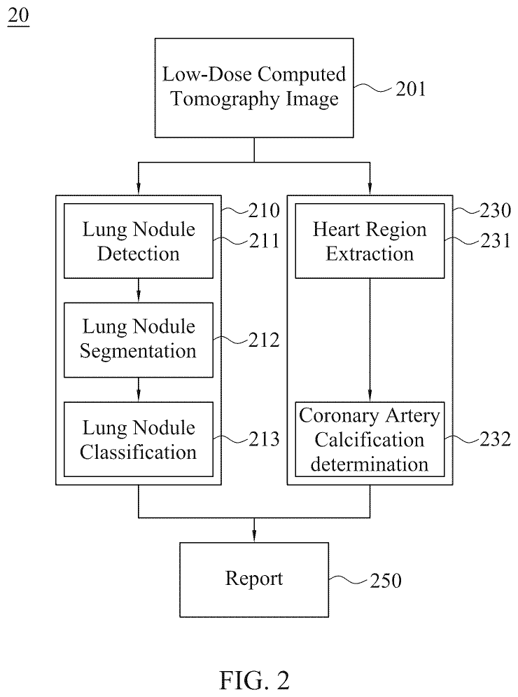

FIG. 2 is a flowchart showing a method 20 of processing a low-dose CT image, in accordance with some embodiments. The method 20 can include operations 201, 210, 230, and 250.

Referring to FIG. 2, the method 20 can begin at the operation 201, in which a low-dose computed tomography (CT) image is received. In some embodiments, the low-dose CT image can be a thoracic image. The CT image can be generated by computed tomography scan. The CT scan is a medical imaging technique used to obtain detailed internal images of the body. In some embodiments, the CT scanners can utilize a rotating X-ray tube and a row of detectors placed in a gantry to measure X-ray attenuations by different tissues inside the body. The multiple X-ray measurements taken from different angles are then processed on a computer using tomographic reconstruction algorithms to produce tomographic (cross-section) images (virtual “slices”) of a body. In some embodiments, the low-dose CT images in operation 201 can be 2D or 3D images.

Regarding the received low-dose chest CT images, the method 20 can then implement four operations 210 and 230 in parallel or sequentially. In some embodiments, the operation 210 includes three steps 211, 212, and 213. The operation 230 includes steps 231 and 232.

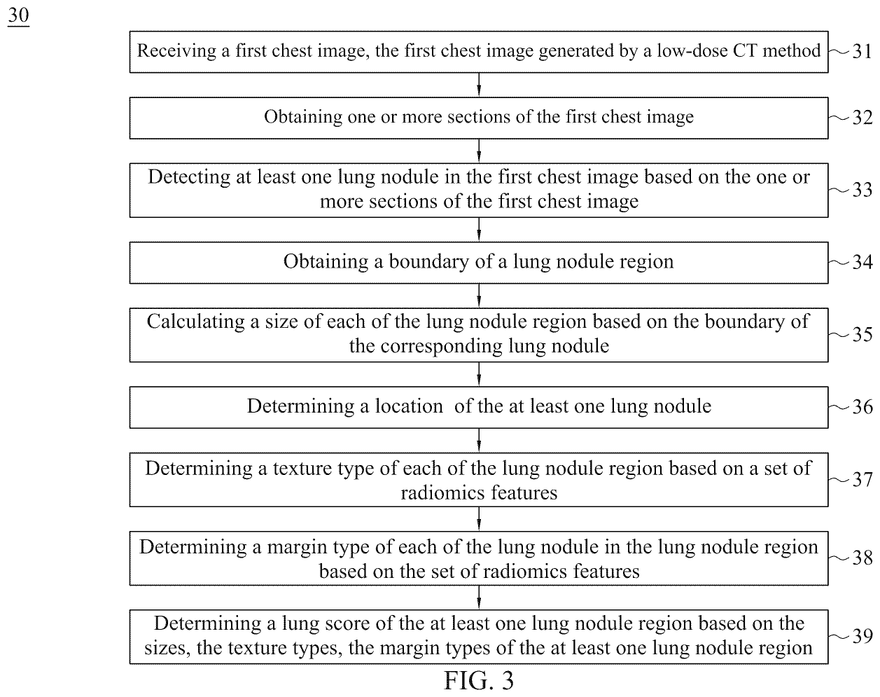

The operation 210 can be a method for processing low-dose CT images to detect and classify lung nodules. The details of the operation 210 can be found in FIGS. 3-11B. In step 211, the lung nodule can be detected in the low-dose CT images. Step 211 constitutes lung nodule detection.

In step 212, a lung nodule region can be determined based on the detected lung nodule. In some embodiments, a boundary of the lung nodule region can be obtained based on semantic segmentation. In some embodiments, the size of the lung nodule region can be calculated. For example, the diameter of the lung nodule region, the longest distance of the lung nodule region, the area of the lung nodule region, and the perimeter of the lung nodule region may be obtained. Step 212 constitutes lung nodule segmentation.

In step 213, the lung nodule region can be classified to determine a nodule score of the lung nodule in the lung nodule region. In some embodiments, the nodule score of the lung nodule can be determined based on a set of radiomics features of the low-dose CT image. The lung nodule score can ascertain the condition of the lung nodule. For example, it can be determined whether the lung nodule is likely to affect lung health. Step 213 constitutes lung nodule classification.

A lung nodule is an abnormal growth formed in a lung. In some embodiments, one lung may have one or more lung nodules. The nodules may develop in one lung or both. Most lung nodules are benign, that is, not cancerous. Rarely, lung nodules may be a sign of lung cancer. The present disclosure can detect and determine whether a CT image captured from a human chest includes lung nodules. Moreover, the present disclosure can further determine whether the detected lung nodules are benign or cancerous. For example, the detected lung nodules can be classified according to the LungRADS, which is an international criteria.

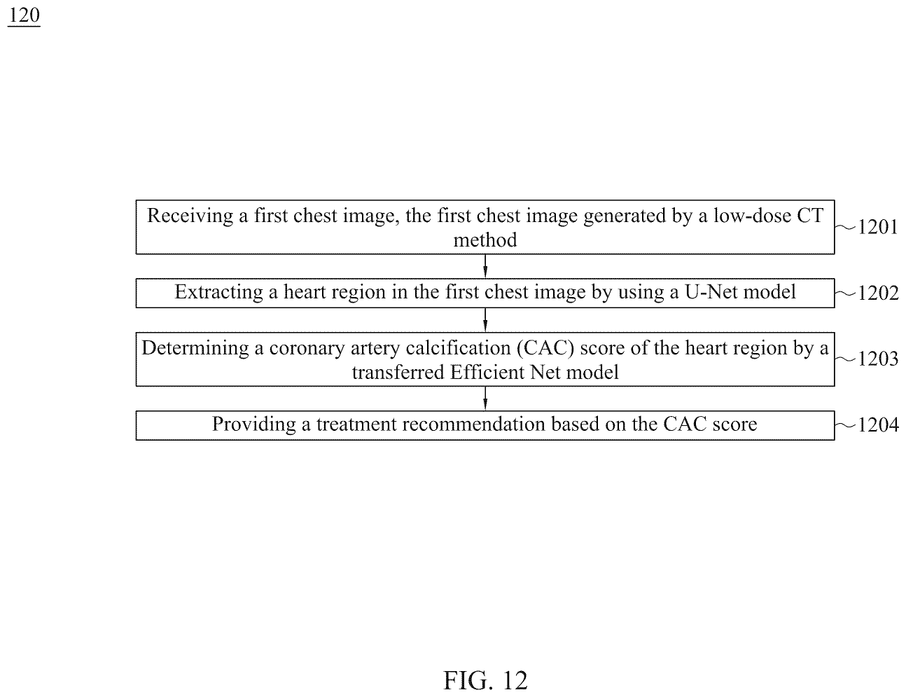

The operation 230 can be a method for processing low-dose CT images to determine a coronary artery calcification (CAC) score. The details of the operation 230 can be found in FIGS. 12-15. In step 231, a heart region can be detected and extracted from the chest CT images. Step 231 constitutes heart region extraction.

In step 232, the CAC score of the heart region can be determined by a model. In some embodiments, the model can be an Efficient Net model. The Efficient Net model can be trained from a pre-trained model for heart full-dose reference CT images and a low-dose reference CT image captured from the same region. In some embodiments, the training of the Efficient Net model can be a transfer learning. Accordingly, the transferred Efficient Net model can determine the CAC score of the heart region of the low-dose CT images. Step 232 constitutes coronary artery calcification determination.

After operations 210 and 230 are performed, operation 250 can be performed to generate a report stating the results of the operations 210 and 230. For example, the report can include the nodule score and location of lung nodules obtained in operation 210, and further include treatment recommendations obtained from a database. The report can include the CAC score obtained in operation 230, and further include treatment recommendations obtained from a database. In some embodiments, the report generated in operation 250 can include one or more results of the operations 210 or 230.

In conventional practice, CAC determinations are obtained from the full-dose CT image. Therefore, the subject disclosure provides a method for determining CAC by processing the low-dose CT images. Compared to full-dose CT images, the low-dose CT images are considerably less radiation exposure.

In addition, the present disclosure provides a method for processing one low-dose CT image (or one set of low-dose CT image) of the chest to determine at least four conditions (i.e., lung nodule and coronary artery calcification). In this case, the subject needs be exposed to the low-dose CT only once while still receiving several examination results.

FIG. 3 is a flowchart showing a method 30 of processing a low-dose CT image to determine a nodule score of lung nodules, in accordance with some embodiments. The method 30 includes operations 31, 32, 33, 34, 35, 36, 37, 38, and 39. In some embodiments, this method 30 can be performed by one or more models. For example, the models can utilize artificial intelligence (AI). In some embodiments, a memory can store instructions, which may be executed by a processor to perform the method 30.

In operation 31, a first chest image can be received. The first chest image is generated by a low-dose CT method. In some embodiments, one or more chest images can be received. The chest image can be a 2D image. In another embodiment, the chest image can be a 3D image. The chest image can include one or more organs. For example, the chest image can include lungs, heart, thoracic vertebrae, ribs, sternum, clavicle, or others.

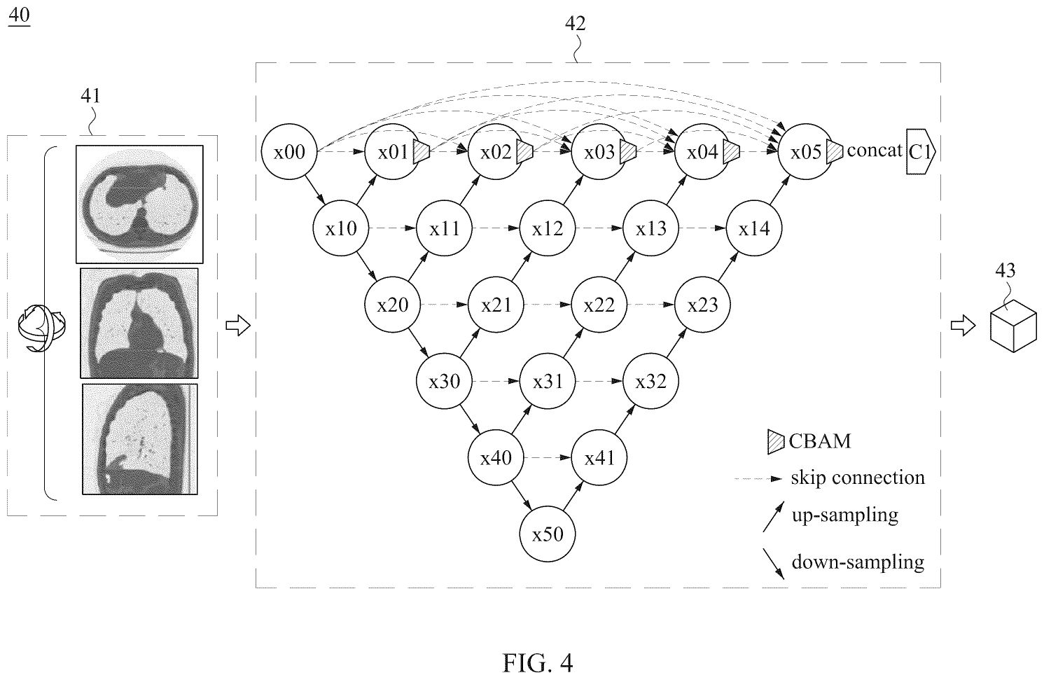

In operation 32, one or more sections of the first chest image can be obtained. In some embodiments, the 3D first chest image can be sectioned along a plane to obtain a 2D section image. In some embodiments, the 3D first chest image can be sectioned along any orientation. In some embodiments, operations 32 and 33 may correspond to operation 211 in FIG. 2. An image process architecture in FIG. 4 discloses embodiments of the operations 32 and 33.

FIG. 4 is a diagram of an image processing architecture 40, in accordance with some embodiments. The image processing architecture 40 includes operations 41, 42, and 43. The operation 41 can correspond to the operation 32. The operations 42 and 43 can correspond to the operation 33.

In operation 41, the first chest image can be sectioned along one or more orientations. For example, the section of the first chest image can be sectioned along a sagittal plane. The section of the first chest image can be sectioned along a coronal plane. The section of the first chest image can be sectioned along an axial plane. The section of the first chest image can be sectioned along a plane inclined +/−30 degrees from the coronal plane to the sagittal plane. The section of the first chest image can be sectioned along a plane inclined +/−30 degrees from the coronal plane to the axial plane. The section of the first chest image can be sectioned along a plane inclined +/−15 degrees from the sagittal plane to the coronal plane. The section of the first chest image can be sectioned along a plane inclined +/−15 degrees from the sagittal plane to the axial plane. In some embodiments, the first chest image can include eleven sections. In another embodiments, the first chest image can include more than eleven sections.

Referring back to FIG. 3, in operation 33, at least one lung nodule in the first chest image can be detected based on the one or more sections of the first chest image. As mentioned, operation 33 can be described in operations 42 and 43 of FIG. 4.

Operation 42 may be performed with a deep learning model. Operation 42 may be performed with an Efficient Net model. In FIG. 4, an exemplary Efficient Net model is shown in operation 42. The one or more sections of the first chest image are input into the Efficient Net model. In some embodiments, part of the sections of the first chest image are input into the Efficient Net model. For example, three sections of the first chest image are input into the Efficient Net model.

The Efficient Net model can process one or more sections of the first chest image, such that a lung nodule in the first chest image can be detected. In some embodiments, the Efficient Net model can process three sections of the first chest image. In another embodiment, the Efficient Net model can randomly select three from the eleven sections and locate lung nodules therein. In some embodiments, the Efficient Net model can be pre-trained according to a set of low-dose CT images.

In operation 42, several convolutions, samplings, and skip-connections are performed. The node xij (i is one of 0, 1, 2, 3, 4, 5; j is one of 0, 1, 2, 3, 4, 5) indicates convolution. The down solid arrow indicates down sampling. The up solid arrow indicates up sampling. The dashed arrow indicates skip connection. For example, the output of the convolution at node x00 is down sampled for the convolution at node x10. The output of the convolution at node x00 is skip-connected to the node x01. The output of the convolution at node 01 is processed by a CBAM (Convolutional Block Attention Module) and then skip-connected to the node x02. The term “concat” in FIG. 4 indicates the concatenation operation. In particular, in the concatenation operation, the outputs of nodes x00, x01, x02, x03, x04, and x05 are concatenated (or stacked together). After the concatenation operation, a convolution is performed on the concatenated data through the convolution layer C1. After the convolution of the convolution layer C1, an output data or an output image would be output to the operation 43.

In operation 43, the output image can include one or more lung nodules marked. In some embodiments, the Efficient Net model can output an image including detected 3D-multi-nodule objects. In some embodiments, the output image can have a size of 64×64×64 pixels.

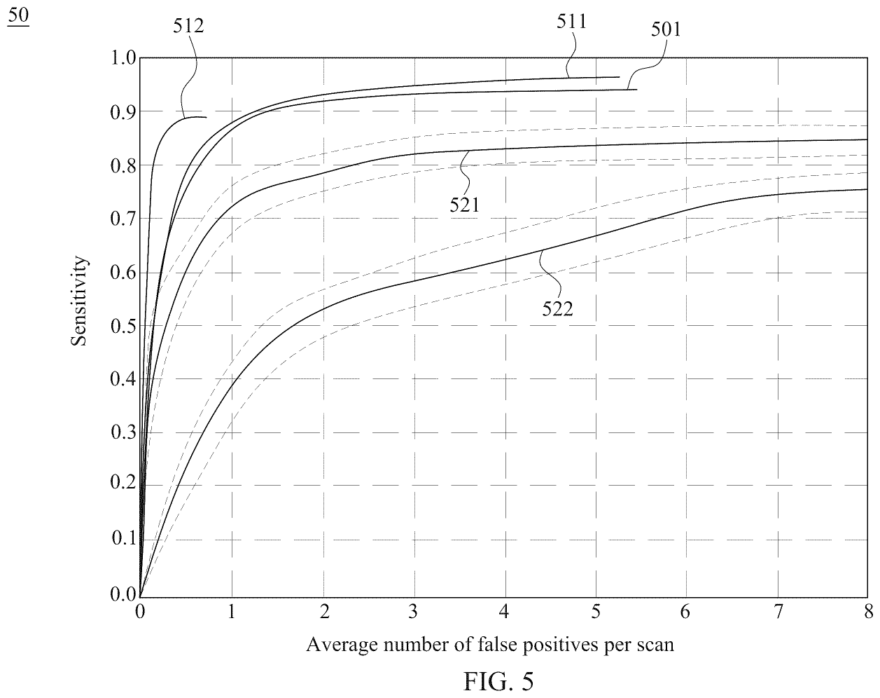

FIG. 5 is a diagram 50 of lung nodule detection, in accordance with some embodiments.

FIG. 5 illustrates the free-response receiver operating characteristic (FROC) curves for lung nodule detection. A FROC diagram can be used to review the sensitivity values of different embodiment under given average numbers of false positives per scan. The number of false positives may be in accordance with the possibility of positive cases and may be adjusted or reduced by adjusting the corresponding thresholds. While adjusting the number of false positives, the number of false negatives may increase and then cause sensitivity value decreased. In theory, FROC curves gradually increases from the left to the right, and undulated variation may not occurs. Therefore, for the best classifier, the corresponding FROC curve would gradually approach to the line of sensitivity equal to 1. The x-axis of FIG. 5 indicates the false positive rate, false positive per scan (FPS), or average number of false positive per scan. The y-axis of FIG. 5 indicates the sensitivity or the true positive rate. FIG. 5 includes curves 501, 511, 512, 521, and 522.

The curve 501 represents the present disclosure. In some embodiments, the curve 501 includes the results of all detected nodules exceeding 3 mm. That is, the detected lung nodules have a diameter exceeding 3 mm. The curve 511 represents a first reference, which is obtained from the ground truth (GT). In some embodiments, the data obtained according to ground truth may indicate that these data is obtained according to the experts' advises. In some embodiments, the curve 511 includes the results of nodules exceeding 5 mm. The curve 512 represents a second reference, which is also obtained from the GT. In some embodiments, the curve 512 includes the results of nodules in a range of 3 to 5 mm. The curve 521 represents a first comparative embodiment, which uses a method different from that of the present disclosure to detect nodules. The dashed lines above and below the curve 521 indicate the possible range of the curve 521. The curve 522 represents a second comparative embodiment, which uses another method different from that of the present disclosure to detect nodules. The dashed lines above and below the curve 522 indicate the possible range of the curve 522.

The area under the curve (AUC) may be used to determine the accuracy of the predictor or the classifier. For example, if the AUC equals 1, the predictor (or the classifier) is perfect, and every prediction is correct. The AUC of the curves 501, 511, 512, 521, and 522 may be used to evaluate the accuracies or prediction performances of the corresponding method. Under a given range (e.g., from 0 to 5) in the x-axis, the AUC of curve 501 is greater than those of curves 521 and 522. It indicates that the corresponding method of the curve 511 (i.e., the method of the present disclosure) is more accurate than those of curves 521 and 522. Under a given range (e.g., from 0 to 5) in the x-axis, the AUC of curve 501 is close to that of curve 511. Curve 511 is obtained from the ground truth for nodules having diameters exceeding 3 mm. That is, the corresponding method of the curve 511 (i.e., the method of the present disclosure) and the ground truth have almost the same accuracy. Therefore, the prediction performance and accuracy for lung nodule detection of the present disclosure is good.

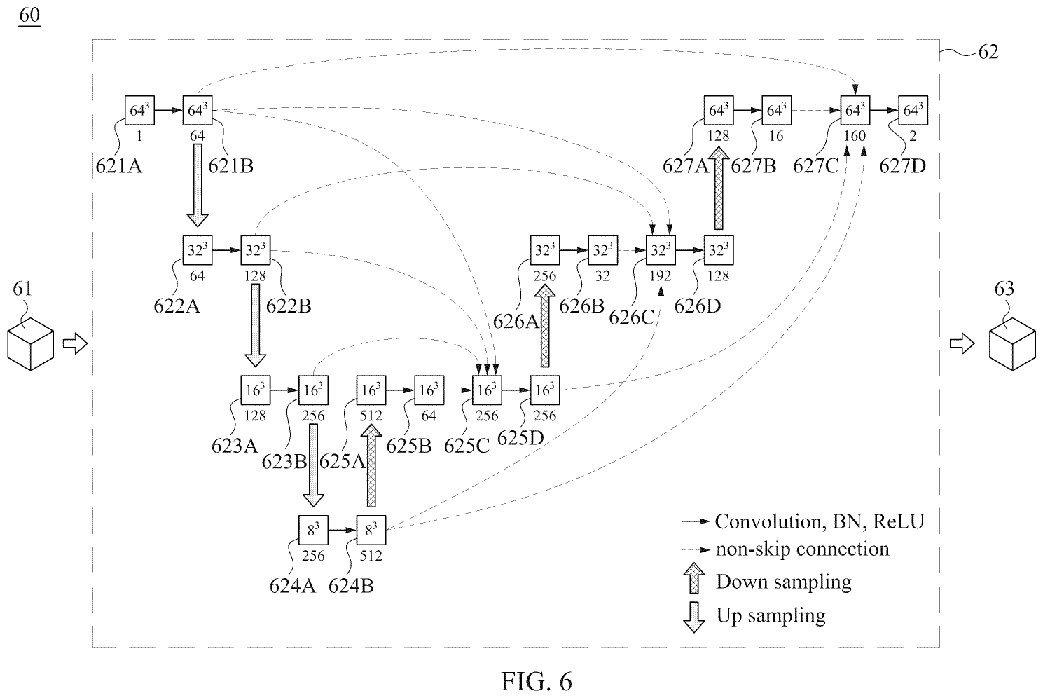

In operation 34, a boundary of lung nodule regions can be obtained based on the at least one lung nodule. The lung nodule regions of the first chest image can be determined based on the at least one lung nodule. In some embodiments, the boundary of the lung nodules can be determined based on a nodule semantic segmentation. The details of the nodule semantic segmentation can be found in FIG. 6.

FIG. 6 is a diagram of an image processing architecture 60, in accordance with some embodiments. The image processing architecture 60 includes operations 61, 62, and 63. The operations 61, 62, and 63 can correspond to the operation 34.

In operation 61, the image output from the operation 43 can be processed and input. In particular, operation 61 identifies one detected nodule in the image from the operation 43 and crops an image centered with the detected nodule. In some embodiments, the cropped image can have a size of 64×64×64 pixels. In some embodiments, multiple cropped images may be generated when multiple modules are detected in the image output from the operation 43.

The operation 62 is performed with a deep learning model. The operation 62 may be performed with a U-Net model. In FIG. 6, the architecture of the U-Net model is shown in operation 62. The image obtained at the operation 43 can be processed and input into the U-Net model.

The U-Net model can process the image, such that boundaries of lung nodule regions can be obtained based on the at least one lung nodule detected in the first chest image. In some embodiments, the U-Net model can be pre-trained according to a set of low-dose CT images. In some embodiments, the U-Net is a convolutional neural network for biomedical image segmentation. The network is based on the fully convolutional network and its architecture was modified and extended to work with fewer training images and to yield more precise segmentations.

Operation 62 in FIG. 6 discloses an exemplary embodiment of the U-Net model. The U-Net model may be a U-Net+++ model. In some embodiments, several data sets are involved in the U-Net model. The data sets may include data 621A, data 621B, data 622A, data 622B, data 623A, data 623B, data 624A, data 624B, data 625A, data 625B, data 625C, data 625D, data 626A, data 626B, data 626C, data 626D, data 627A, data 627B, data 627C, and data 627D.

Data 621A may be the input image of the U-Net model. Data 621A may be an image having a size of 64×64×64 (e.g., 64³) pixels, which is cropped from a low-dose CT image and centered with the detected nodule. Data 621A may have 1 channel. Data 621B is generated from data 621A through the calculations of convolution, BN (batch normalization), and ReLU (rectified linear unit). Data 621B has 64 channels, each channel has a size of 64×64×64 (e.g., 64³) pixels.

Data 622A is generated from data 621B through the calculations of down sampling. The down sampling may be performed by max-pooling. Data 622A has 64 channels, each channel has a size of 32×32×32 (e.g., 32³) pixels.

Data 625C is generated from data 625B, data 623B, data 622B, and data 621B through the non-skip connection of data 625B, data 623B, data 622B, and data 621B. Because the size of data 625B (i.e., 16³) is different from that of data 621B (i.e., 64³), data 621B may be down sampled (e.g., by max-pooling) before the non-skip connection. Because the size of data 625B (i.e., 16³) is different from that of data 622B (i.e., 32³), data 622B may be down sampled (e.g., by max-pooling) before the non-skip connection. After the non-skip connection, data 625C has 256 channels, each channel has a size of 16×16×16 (e.g., 16³) pixels.

Data 626A is generated from data 625D through the calculations of up sampling. Data 626A has 256 channels, each channel has a size of 32×32×32 (e.g., 32³) pixels.

Data 627D is generated from data 627C through the calculations of convolution, BN, and ReLU. Data 627D has 2 channels, each channel has a size of 64×64×64 (e.g., 64³) pixels. One channel of data 627D may be identical to the input image (e.g., a low-dose CT image of chest), and the other channel of data 627D may be a mask to the input image that indicates the region of one nodule.

In operation 63, the output image can include one lung nodules having boundaries thereof being determined. In some embodiments, the output images can have a size of 64×64×64 pixels.

In some embodiments, the image processing architecture 60 may be performed multiple times when multiple modules are detected in the image output from the operation 43.

Referring back to FIG. 3, in operation 35, a size (or a maximum diameter) of each of the lung nodule regions can be calculated based on the boundary of the corresponding lung nodule. For example, the diameter of the lung nodule region, the longest length of the lung nodule region, the area of the lung nodule region, the perimeter of the lung nodule region may be obtained based on the nodule semantic segmentations.

In some embodiments, the operations 34 and 35 correspond to the operation 212 in FIG. 2.

In operation 36, a location of the lung nodules can be determined. The location of the lung nodules can be determined based on an image including detected 3D-multi-nodule objects obtained in operation 43. The location of the lung nodules can be determined based on a set of radiomics features. In some embodiments, the location of the lung nodules can be determined based on a set of radiomics features and a set of slice features. In some embodiments, the location of the lung nodule can include a right upper lobe (RUL), a right middle lobe (RML), a right lower lobe (RLL), a left upper lobe (LUL), a left lower lobe (LLL), and a lingular lobe. The location of the lung nodules can be determined based on coordinates in each section image of the first chest.

In some embodiments, the set of radiomics features can be extracted from merely the region of interest (ROI) or volume of interest (VOI). The ROI and VOI can be the determined lung region in the chest low-dose CT images. In some embodiments, the region of interest (ROI) or volume of interest (VOI) may be extracted or calculated from an image including detected 3D-multi-nodule objects obtained in operation 43. In some embodiments, the region of interest (ROI) or volume of interest (VOI) may be extracted or calculated from one or more images obtained in operation 63. In some embodiments, the set of radiomics features can be extracted or calculated the region of interest (ROI) or volume of interest (VOI).

In some embodiments, the set of radiomics features can be extracted or calculated from an image including detected 3D-multi-nodule objects obtained in operation 43. The set of radiomics features can be extracted or calculated from one or more images obtained in operation 63. The set of radiomics features can be extracted or calculated from the region of interest (ROI) or volume of interest (VOI). The set of radiomics features can include gray-level co-occurrence matrices (GLCM) textures, grey-level run-length matrix (GLRLM) textures, gray level size zone matrix (GLSZM) textures, neighbouring gray tone difference matrix (NGTDM) textures, and gray-level difference matrix (GLDM) textures. In some embodiments, the set of slice features can include slice information of segmentation of nodules (SISN).

In operation 37, a texture type of each of the lung nodules can be determined with Computed Tomography to Report (CT2Rep) model. A texture type of each lung nodules can be determined based on a set of radiomics features. A texture type of each lung nodules can be determined based on a set of slice features. In some embodiments, the set of radiomics features may have 107 units. The set of radiomics features can be extracted or calculated from the first chest image. In some embodiments, the texture type can include solid, sub-solid, and ground-glass opacity (GGO).

In operation 38, a margin type of each of the lung nodules can be determined with Computed Tomography to Report (CT2Rep) model. A texture type of each lung nodules can be determined based on a set of radiomics features. A texture type of each lung nodules can be determined based on a set of slice features. In some embodiments, the set of radiomics features may have 107 units. The set of radiomics features can be extracted or calculated from the first chest image. In some embodiments, the margin type can include sharp circumscribed, lobulated, indistinct, and speculated.

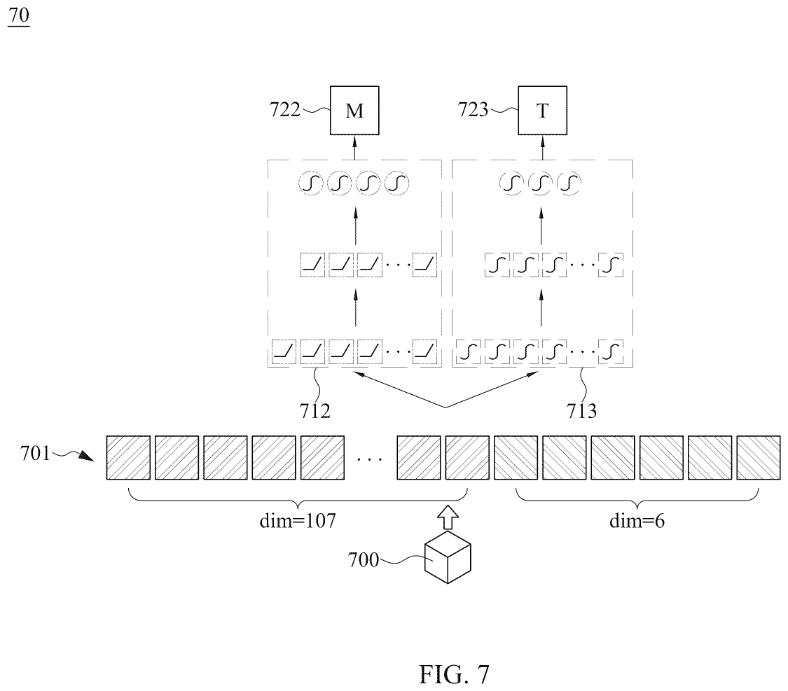

The details of the texture type and margin type determination according to some embodiments of the present disclosure can be found in FIG. 7.

In the CT2Rep model, the 107 units of the radiomics features may be extracted or calculated from the chest image and/or the regions of the nodules (e.g., the region of interest (ROI) or volume of interest (VOI)), the 107 units of the radiomics features then input to the CT2Rep model

FIG. 7 is a diagram of a classification framework 70 of features of an image, in accordance with some embodiments. In some embodiments, the classification framework 70 may be regarded as a CT2Rep model. The classification framework 70 includes one or more input images 700, a set of features 701, operations 712 and 713, a margin result 722, and a texture result 723.

The classification procedure 70 can have input images 700. In some embodiments, the input images 700 may include a low-dose (LD) CT image of chest and the regions of nodules (e.g., the ROI or VOI), which may be the images obtained at operation 62 or 63. In some embodiments, the input images 700 may be an image including detected 3D-multi-nodule objects (e.g., the image obtained at operation 43).

A set of features 701 can be extracted or calculated from the regions of nodules (i.e., ROI or VOI). A set of features 701 may be extracted calculated from the low-dose (LD) CT image of chest and the regions of nodules (e.g., the ROI or VOI). In some embodiments, the set of features 701 can include a set of radiomics features and a set of slice features. In some embodiments, the ratio of labeled slices to total slices and some other related slicing information indicated the location of the nodules to a certain extent. Therefore, a total of six features were extracted from the slice information of segmentation of nodules (SISN) and were used in the present disclosure.

In some embodiments, the number of the radiomics features can be different from the number of the slice features. The number of the radiomics features can exceed that of the slice features. In one embodiment, the set of radiomics features can include 107 features. In some embodiments, the set of slice features can include 6 features.

The set of radiomics features can be classified as two main groups: first-order and second-order. In some embodiments, the first-order features are related to the characteristics of intensity distribution in the VOL. For example, the intensity distribution features can include 18 features. In another embodiment, the first-order features are related to the shape-based 2D and 3D morphological features of the VOI. For example, the shape-based features can include 14 features.

Alternatively, the second-order features can be regarded as a textural analysis, providing a measure of intra-lesion heterogeneity and further assessing the relationships between the pixel values within the VOL. The second-order features can include gray level co-occurrence matrix (GLCM), gray level run length matrix (GLRLM), gray level size zone matrix (GLSZM), neighboring gray-tone difference matrix (NGTDM), and gray level dependence matrix (GLDM). In some embodiments, the GLCM can include 24 features. The GLRLM can include 16 features. The GLSZM can include 16 features. The NGTDM can include 5 features. The GLDM can include 14 features.

The operations 712 and 713 can constitute multi-objective deep learning processes.

Referring to FIG. 7, the operations 712 and 713 can include an input layer in the bottom, two dense layers above the input layer, and an output layer. In some embodiments, the set of features 701 would be used for the input layer in operations 712 and 713. That is, the operations 712 and 713 can process the set of radiomics features and/or the set of slice features.

For operation 712, the set of features can be processed in the input layer and then output to the first dense layer and the second dense layer. During the activation of the dense layers, the features can be further processed with dropout. After the activation of the two dense layers, the features can be output to the output layer. Completing the whole multi-objective deep learning model (e.g., a Support Vector Machine (SVM)) in operation 712, a margin result 722 can be obtained. In some embodiments, the margin result 722 can be determined based on merely the set of radiomics features. In some embodiments, the margin result 722 can include sharp circumscribed, lobulated, indistinct, and speculated.

For operation 713, the set of features can be processed in the input layer and then output to the first dense layer and the second dense layer. During the activation of the dense layers, the features can be further processed with dropout. After the activation of the two dense layers, the features can be output to the output layer. Completing the whole multi-objective deep learning model (e.g., a Support Vector Machine (SVM)) in operation 713, a texture result 723 can be obtained. In some embodiments, the texture result 723 can be determined based on merely the set of radiomics features. In some embodiments, the texture result 723 can include solid, sub-solid, and ground-glass opacity (GGO).

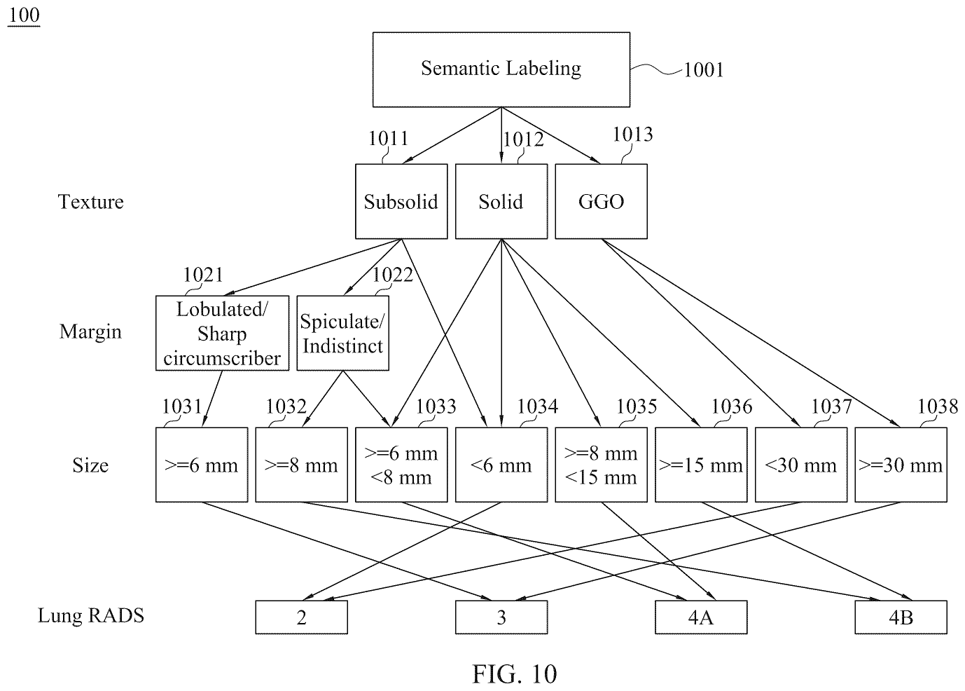

A nodule score of the lung nodule can be determined based on the sizes, the texture result 723, and the margin result 723 of the lung nodule. In some embodiments, the nodule score can be acted as Lung-RADS (Lung Imaging Reporting and Data System) score. The details of the nodule score classification can be found in FIG. 10.

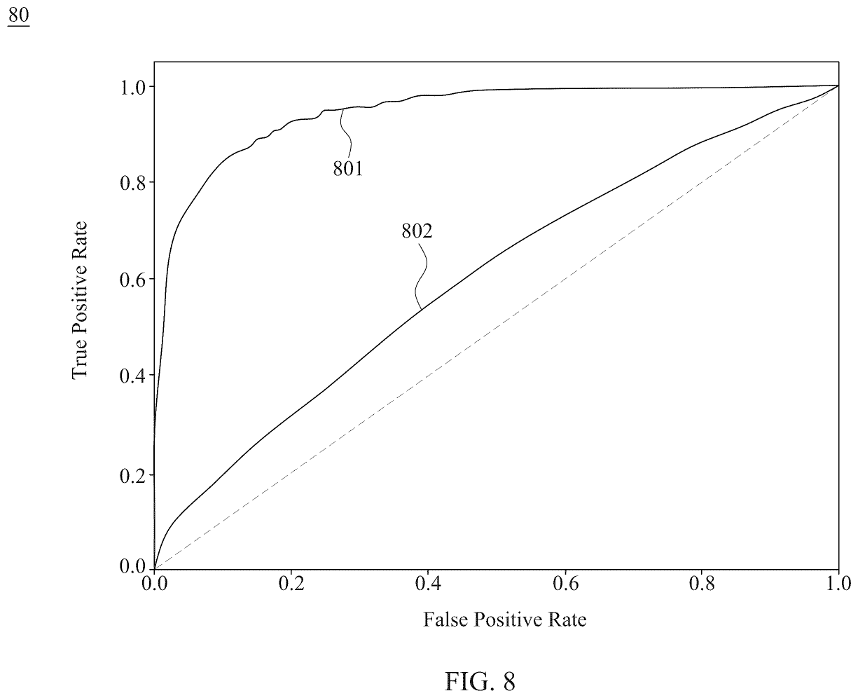

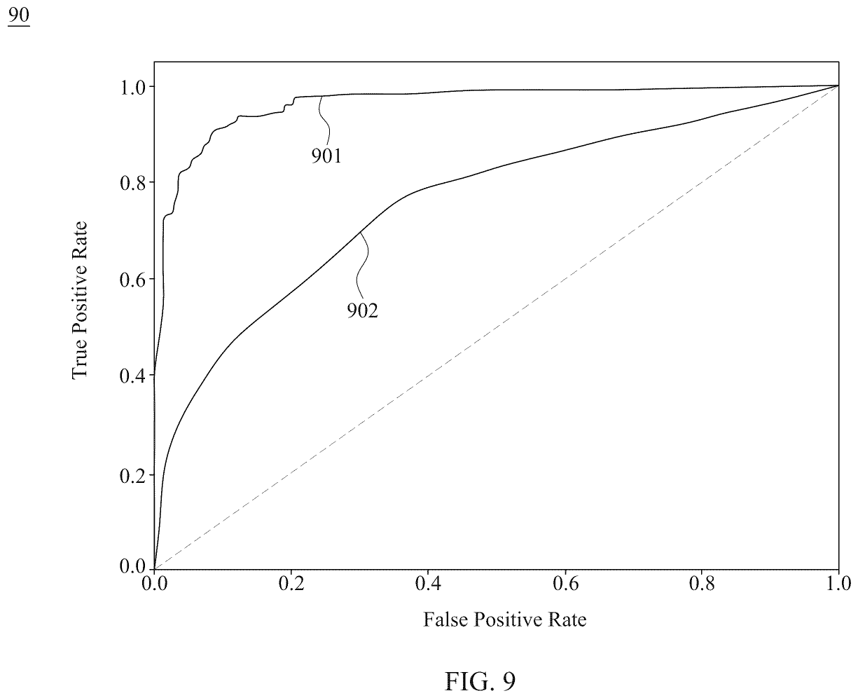

FIGS. 8 and 9 are diagrams of performance, in accordance with some embodiments.

FIG. 8 illustrates the receiver operating characteristic (ROC) curve for margin of the lung nodules. The x-axis of FIG. 8 indicates the false positive rate. The y-axis of FIG. 8 indicates the sensitivity or the true positive rate. FIG. 8 includes curves 801 and 802. The curve 801 represents the present disclosure. The curve 802 represents a comparative embodiment. The area under the curve (AUC) may be used to determine the accuracy of the predictor or the classifier. For example, if the AUC equals 1, the predictor (or the classifier) is perfect, and every prediction is correct. In FIG. 8, the AUC is 0.95 for the curve 801, and the AUC is 0.60 for the curve 802. From FIG. 8, the prediction performance for margin of the lung nodules of the present disclosure is good.

FIG. 9 illustrates the receiver operating characteristic (ROC) curve for texture of the lung nodules. The x-axis of FIG. 9 indicates the false positive rate. The y-axis of FIG. 9 indicates the sensitivity or the true positive rate. FIG. 9 includes curves 901 and 902. The curve 901 represents the present disclosure. The curve 902 represents a comparative embodiment. In FIG. 9, the AUC is 0.97 for the curve 901, and the AUC is 0.76 for the curve 902. From FIG. 9, the prediction performance for texture of the lung nodules of the present disclosure is good.

In operation 39, a nodule score of the lung nodule can be determined based on size, texture type, and margin type of the at least one lung nodule. In some embodiments, the nodule score can be Lung-RADS, which is an international criteria to classify the level of lung nodules. In some embodiments, the operations 36, 37, 38, and 39 correspond to the operation 213 in FIG. 2. The details of the nodule score classification can be found in FIG. 10.

FIG. 10 is a diagram of a nodule score classification procedure 100 of features of an image, in accordance with some embodiments. The nodule score classification procedure 100 includes three determining steps depending on the texture, margin, and size. The nodule score classification procedure 100 can include an input 1001, texture types 1011, 1012, and 1013, margin types 1021 and 1022, size ranges 1031, 1032, 1033, 1034, 1035, 1036, 1037, and 1038, and nodule scores 1041, 1042, 1043, 1044. In some embodiments, the nodule score classification procedure 100 can be performed by the CT2Rep model.

The nodule score classification procedure 100 can assess texture type, margin type, and then size, such that a nodule score (i.e., Lung RADS) can be determined.

The nodule score classification procedure may begin from the semantic labeling 1001. The semantic labeling 1001 is the data obtained from the chest LDCT images. In some embodiments, the semantic labeling 1001 can include the size of the lung nodules obtained in operation 35, the location of the lung nodules obtained in operation 36, the texture type of the lung nodules obtained in operation 37, and the margin type of the lung nodules obtained in operation 38.

First, the semantic labeling 1001 can be classified according to the texture type. The texture type can be classified as sub-solid 1011, solid 1012, and GGO 1013.

Second, the semantic labeling 1001 can be classified according to the margin type. The margin type can be classified as lobulated/sharp circumscriber 1021 and speculate/indistinct 1022. Although the margin types have four different types, they can be classified into the two groups based on severity of lung nodule.

Third, the semantic labeling 1001 can be classified according to size range. Size range 1031 corresponds to lung nodules exceeding 6 mm. Size range 1032 corresponds to lung nodules exceeding 8 mm. Size range 1033 corresponds to lung nodules from 6 to 8 mm. Size range 1034 corresponds to lung nodules under 6 mm. Size range 1035 corresponds to lung nodules from 8 to 15 mm. Size range 1036 corresponds to lung nodules exceeding 15 mm. Size range 1037 corresponds to lung nodules under 30 mm. Size range 1038 corresponds to lung nodules exceeding 30 mm.

In some embodiments, the Lung RADS can include four levels, i.e., levels 2, 3, 4A, and 4B. Lung RADS increases with lung nodule severity.

In some embodiments, if the texture type is determined as solid 1012 or GGO 1013, the margin type needs not be determined. When the texture type of lung nodule is determined as GGO 1013, the size thereof can be classified as greater or less than 30 mm. With the texture type of GGO 1013, the lung nodule having a size exceeding 30 mm can be classified as Lung RADS level 3. Lung nodules with texture type of GGO 1013 having a size less than 30 mm can be classified as Lung RADS level 2.

When the texture type of lung nodule is determined as solid 1012, the nodule score thereof can be classified as Lung RADS levels 4A, 2, 4A, and 4B according to size ranges 1033, 1034, 1035, and 1036, respectively.

When the texture type of lung nodule is determined as sub-solid 1011, the nodule score thereof can be classified as Lung RADS level 2 with the size less than 6 mm. For those exceeding 6 mm having sub-solid texture, the margin type of the lung nodule must be determined. The lung nodule having lobulated/sharp circumscriber 1021 and the size exceeding 6 mm can be classified as Lung RADS level 3. With the margin type of speculate/indistinct 1022, the lung nodule exceeding 8 mm can be classified as Lung RADS level 4B. Lung nodules with margin type of speculate/indistinct 1022 in a range of 6 to 8 mm can be classified as Lung RADS level 4A.

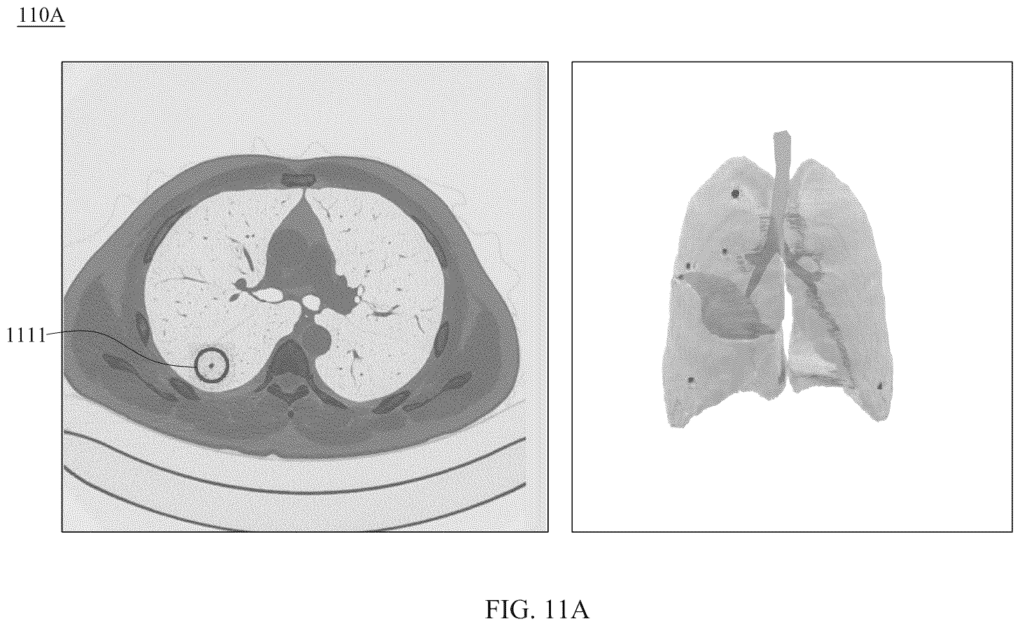

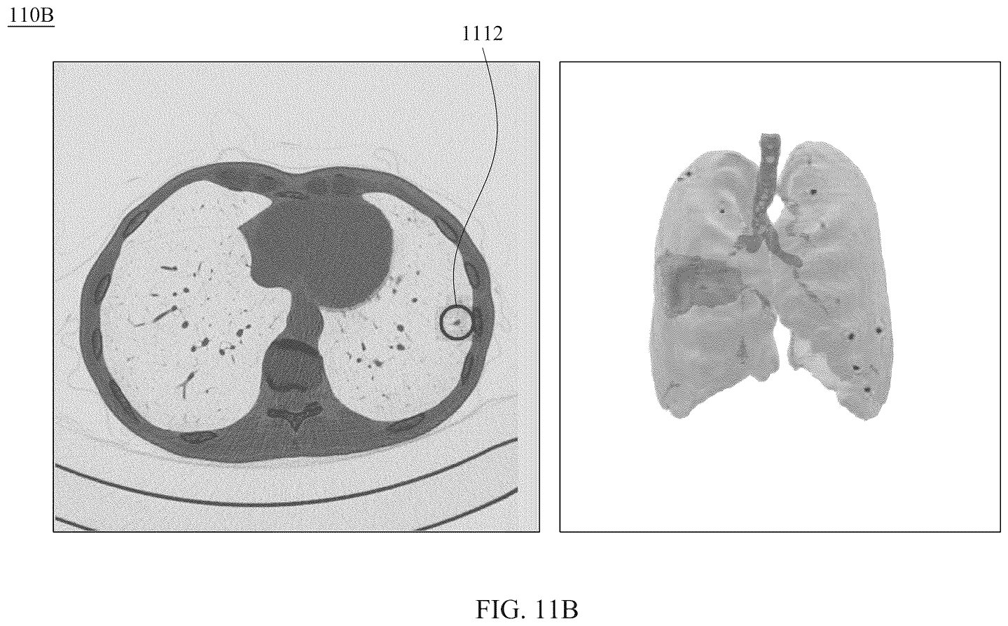

FIGS. 11A and 11B show lung images, in accordance with some embodiments.

FIG. 11A includes an exemplary 2D LDCT image and an exemplary 3D LDCT image generated by the method 30 in FIG. 3. The 2D LDCT image and the 3D LDCT image includes several lung nodules (such as the one in a circle 1111). In some embodiments, the black spots in the 3D LDCT images are the lung nodules.

FIG. 11B includes another exemplary 2D LDCT image and another exemplary 3D LDCT generated by the method 30 in FIG. 3. The 2D LDCT image and the 3D LDCT image includes several lung nodules (such as the one in a circle 1112). In some embodiments, the black spots in the 3D LDCT images are the lung nodules.

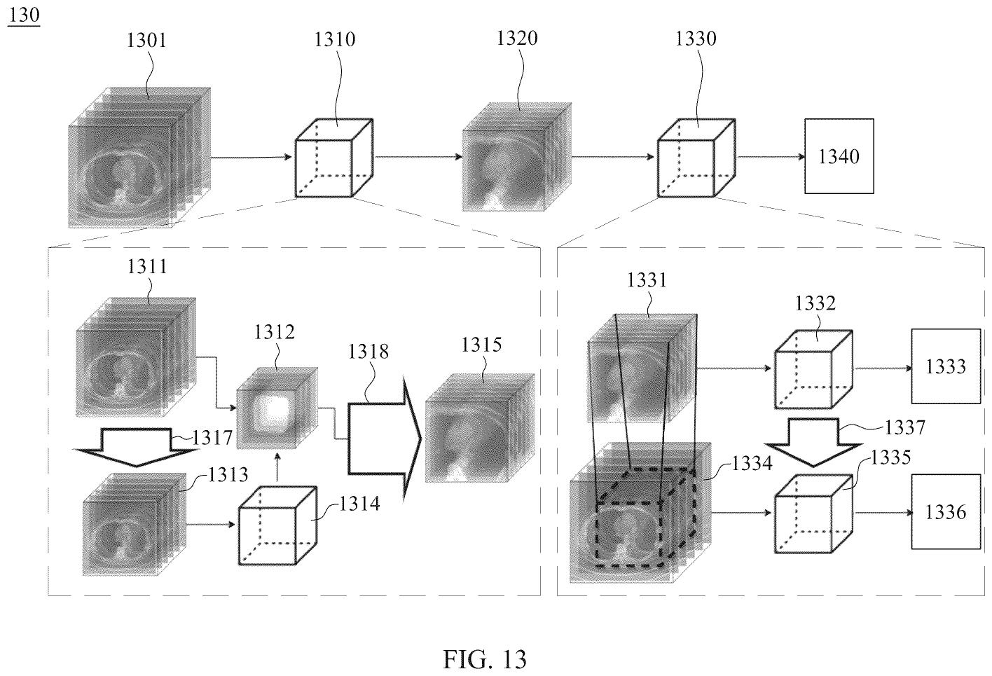

FIG. 12 is a flowchart showing a method 120 of processing a low-dose CT image to determine a coronary artery calcification (CAC) score, in accordance with some embodiments. The method 120 includes operations 1201, 1202, 1203, and 1204. In some embodiments, this method 120 can be performed by one or more models. For example, the models can be artificial intelligence (AI) models. In some embodiments, a memory can store instructions, which may be executed by a processor to perform the method 120. The details of the method 120 can be found in FIG. 13 for better understanding.

In operation 1201, a first chest image can be received. The first chest image is generated by a low-dose CT method. In some embodiments, one or more chest images can be received. The chest image can be a 2D image. In another embodiment, the chest image can be a 3D image. The chest image can include one or more organs. For example, the chest image can include lungs, heart, thoracic vertebrae, ribs, sternum, clavicle, or others.

In operation 1202, a heart region in the first chest image can be extracted by using a U-Net model. The U-Net model is a deep learning model. In some embodiments, the extraction of the heart region can include detecting the heart in the first chest image. In some embodiments, the extraction of the heart region can include determining a boundary of the heart region based on a semantic segmentation. The heart can be detected and the heart region can be determined and extracted. In some embodiments, the location of the heart region can be determined.

In operation 1203, a coronary artery calcification (CAC) score of the heart region can be determined by a transferred Efficient Net model. Coronary artery calcification is an indicator of coronary artery disease so as to control cardiovascular risk. In some embodiments, the transferred Efficient Net model can be trained from a pre-trained model for heart full-dose reference CT images and a low-dose reference CT image captured from a same region.

The pre-trained model is trained by a plurality of heart full-dose reference CT images. For example, the pre-trained model can be trained with 1221 heart full-dose reference CT images. Accordingly, the pre-trained model is ready for determining CAC score based on the full-dose CT images. The pre-trained model can be further trained by a plurality of heart low-dose reference CT images to be the transferred Efficient Net model. For example, the transferred Efficient Net model can be trained from the pre-trained model with 1221 heart low-dose reference CT images. Such model training may be known as transfer learning. Accordingly, the transferred Efficient Net model can analyze the low-dose CT images and determine the CAC score of the heart region in the low-dose CT images.

In operation 1204, a treatment recommendation based on the CAC score can be provided. In some embodiments, the treatment recommendation can be obtained from a database. The treatment recommendation can correspond to difference level of CAC. The level of CAC can be determined based on the CAC score. In some embodiments, the treatment recommendation may provide guideline for patient to understand what to do and what to avoid.

FIG. 13 is a diagram of a CAC determination procedure, in accordance with some embodiments.

FIG. 13 is a diagram of a CAC determination procedure 130, in accordance with some embodiments. The CAC determination procedure 130 includes operations 1301, 1310, 1320, 1330, and 1340. The operation 1301 can correspond to the operation 1201. The operations 1310 and 1320 can correspond to the operation 1202. The operation 1330 can correspond to the operation 1203. The operation 1340 can correspond to the operation 1203.

In operation 1301, one or more chest images can be received. The chest images are generated by a low-dose CT method. The chest images can be 2D images. In another embodiment, the chest image can be 3D images. The chest image can include one or more organs. For example, the chest image can include lungs, heart, thoracic vertebrae, ribs, sternum, clavicle, or others.

In operation 1310, the heart region can be detected and extracted. In operation 1310, heart localization and heart VOI extraction are performed to obtain images of heart. The operation 1310 can include one or more chest low-dose CT (LDCT) images 1311, extracted regions 1312, low resolution LDCT images 1313, a model 1314, and output images 1315.

In some embodiments, the one or more chest LDCT images 1311 can be received. A down sampling operation 1317 can be performed to transform the one or more chest LDCT images 1311 to be low resolution LDCT images 1313. The low resolution LDCT images 1313 can be analyzed easier with smaller file size and less complexity.

The low resolution images 1313 can be input to the model 1314. In some embodiments, the model 1314 can be U-Net model. The U-Net model is a deep learning model. The heart region can be extracted from the low resolution LDCT images 1313 by the model 1314, such that the extracted regions 1312 can be obtained. In some embodiments, the extraction of the heart region can include detecting the heart. In some embodiments, the extraction of the heart region can include determining a boundary of the heart region based on a semantic segmentation. The heart region can be detected, determined, and extracted. In some embodiments, the location of the heart region can be determined.

The extracted regions 1312 can be mapped to original resolution chest LDCT images 1311, such that the output images 1315 can be obtained. The output images 1315 can have the resolution identical to that of the chest LDCT images 1311. In some embodiments, after the mapping operation 1318, the extracted region 1312 can be transformed to the output images 1315 having higher resolution.

In some embodiments, the output images 1315 can be output at operation 1320. The output images 1315 can include the heart region being determined. In some embodiments, the location of the heart region can be determined in the output images 1315.

In operation 1330, a coronary artery calcification (CAC) score of the heart region can be determined by a transferred Efficient Net model. The operation 1530 may involve one or more heart CT images 1331, a pre-trained model 1332, an output 1333, one or more chest LDCT 1334, a transferred Efficient Net model 1335, and an output 1336.

In some embodiments, one or more heart CT images 1331 can be input to the pre-trained model 1332. The heart CT images 1331 can be full-dose CT images. The pre-trained model 1332 can be trained by the heart CT images 1331. For example, the pre-trained model 1332 can be trained with 1221 heart CT images 1331. Accordingly, the pre-trained model 1332 is ready for determining CAC score based on the full-dose CT images.

In some embodiments, the output 1333 can include the CAC score of the heart region of the heart CT images. The output 1333 can be a report showing the CAC score. In some embodiments, the output 1333 can include the treatment recommendation corresponding to the CAC score. The output 1333 can include CAC level according to the CAC score. For example, the risk level 1 represents the CAC score less than 1. The risk level 2 represents the CAC score in a range of 1 to 10. The risk level 3 represents the CAC score in a range of 11 to 100. The risk level 4 represents the CAC score in a range of 101 to 400. The risk level 5 represents the CAC score exceeding 400.

One or more chest LDCT images 1334 can correspond to the one or more heart CT images 1331. In some embodiments, the chest LDCT images 1334 can have the same or similar heart regions as those of the heart CT images 1331. The chest LDCT images 1334 may be the output images 1315 or the images output at operation 1320.

The transferred Efficient Net model 1335 can be trained or obtained based on the pre-trained model 1332. In some embodiments, the transferred Efficient Net model 1335 can be trained or obtained based on a pre-trained model for heart full-dose reference CT images 1331 and chest LDCT images 1334 having the same or similar heart regions. The transferred Efficient Net model 1335 can be obtained by training the pre-trained model 1332 with 1221 chest LDCT images 1334. Such model training method may be known as transfer learning 1337. Once the transfer learning 1337 is completed, the transferred Efficient Net model 1335 can be used to analyze the chest LDCT images 1334 and determine the CAC score of the heart region in the chest LDCT images 1334.

In some embodiments, the output 1336 can include the CAC score of the heart region of the chest LDCT images 1334. The output 1336 can be a report showing the CAC score. In some embodiments, the output 1336 can include the treatment recommendation corresponding to the CAC score. The output 1336 can include CAC risk level according to the CAC score. For example, the risk level 1 represents the CAC score less than 1. The risk level 2 represents the CAC score in a range of 1 to 10. The risk level 3 represents the CAC score in a range of 11 to 100. The risk level 4 represents the CAC score in a range of 101 to 400. The risk level 5 represents the CAC score exceeding 400. In some embodiments, the outputs 1333 and 1336 can be compared to confirm whether the transferred Efficient Net model 1335 is well trained.

After the transferred Efficient Net model 1335 is well trained, the output images 1315, which are LDCT images, can be analyzed through the transferred Efficient Net model 1335, such that the CAC score of the heart region can be determined. The CAC score of the heart region can be output at operation 1340.

In operation 1340, the output can include the CAC score of the heart region of the chest LDCT images. In some embodiments, the output can include the treatment recommendation corresponding to the CAC score. The output can include risk level according to the CAC score. For example, the low risk represents the CAC score less than 10. The moderate risk represents the CAC score in a range of 10 to 100. The high risk represents the CAC score exceeding 100.

The present disclosure provides a method for processing LDCT images to determine the CAC score. Compared to conventional practice, the present disclosure provides the same effect with lower radiation impact. Having the transferred Efficient Net model, the LDCT images can be analyzed, and the CAC score can be determined based on the heart region in the LDCT images. In addition, the report including treatment recommendations can be generated automatically. In this case, since the CAC related report can be generated automatically, manpower burdens are decreased.

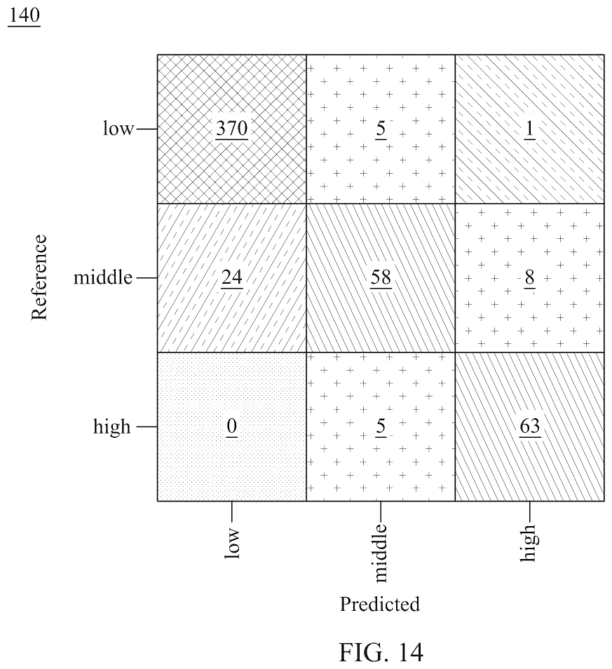

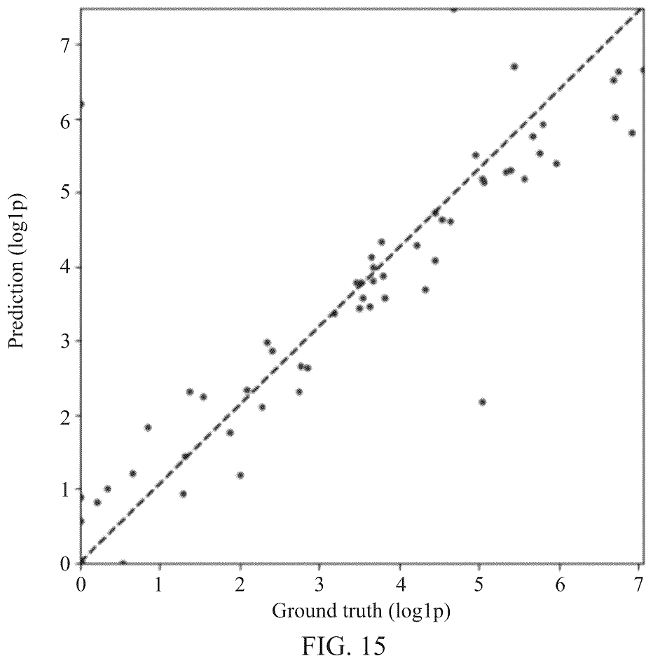

FIGS. 14 and 15 are diagrams of performance, in accordance with some embodiments. FIG. 14 illustrates the confusion matrix 140 of CAC score without normalization. In FIG. 14, the x-axis indicates the predicted CAC score. The y-axis indicates the reference CAC score. In some embodiments, the reference CAC score can be the actual CAC score. FIG. 14 shows that, for the same subject/patient, the predicted CAC score and the actual CAC score have highly positive correlation. That is, the predicted CAC scores according to the present disclosure have high accuracies.

FIG. 15 illustrates the linear regression diagram of CAC score. The x-axis indicates the ground truth of CAC score. In some embodiments, the ground truth can be the actual CAC score of the patient. The y-axis indicates the prediction of CAC score. In some embodiments, the prediction of CAC score can be determined according to the present method (for example, the method shown in FIG. 12). FIG. 15 shows that, for the same subject/patient, the predicted CAC score and the actual CAC score for the same CAC score have highly positive correlation. That is, the predicted CAC scores according to the present disclosure have high accuracies.

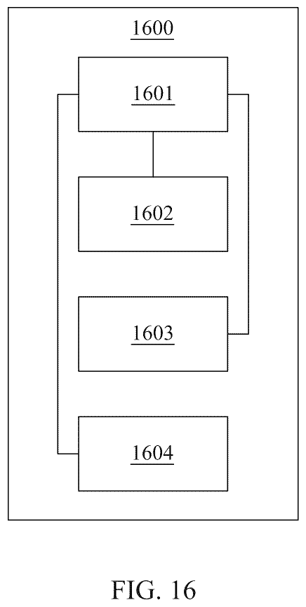

FIG. 16 illustrates a schematic diagram showing a computer device 1600 according to some embodiments of the present disclosure. The computer device 1600 may be capable of performing one or more procedures, operations, or methods of the present disclosure. The computer device 1600 may be a server computer, a client computer, a personal computer (PC), a tablet PC, a set-top box (STB), a personal digital assistant (PDA), a cellular telephone, or a smartphone. The computing device 1600 comprises processor 1601, input/output interface 1602, communication interface 1603, and memory 1604. The input/output interface 1602 is coupled with the processor 1601. The input/output interface 1602 allows the user to manipulate the computing device 1600 to perform the procedures, operations, or methods of the present disclosure (e.g., the procedures, operations, or methods disclosed in FIGS. 2-4, 6, 7, 10, 12, and 13). The communication interface 1603 is coupled with the processor 1601. The communication interface 1603 allows the computing device 1600 to communicate with data outside the computing device 1600, for example, receiving data including images and/or any essential features. A memory 1604 may be a non-transitory computer readable storage medium. The memory 1604 is coupled with the processor 1601. The memory 1604 has stored program instructions that can be executed by one or more processors (for example, the processor 1601).

For example, upon execution of the program instructions stored on the memory 1604, the program instructions cause performance of the one or more procedures, operations, or methods disclosed in the present disclosure. For example, the program instructions may cause the computing device 1600 to perform, for example, receiving a LDCT image of chest; detecting, by the processor 1601, at least one lung nodule in the LDCT image; determining, by the processor 1601, at least one lung nodule region of the LDCT image based on the at least one lung nodule; and classifying, by the processor 1601, the at least one lung nodule region based on a first set of radiomics features of the at least one lung nodule region of the LDCT image to obtain a nodule score of the at least one lung nodule in the lung nodule region.

For example, upon execution of the program instructions stored on the memory 1604, the program instructions cause performance of the one or more procedures, operations, or methods disclosed in the present disclosure. For example, the program instructions may cause the computing device 1600 to perform, for example, receiving a LDCT image of chest; detecting, by the processor 1601, at least one lung nodule in the LDCT image; extracting, by the processor 1601, a heart region in the LDCT image by using a U-Net model; and determining, by the processor 1601, a coronary artery calcification (CAC) score of the heart region by an transferred Efficient Net model.

The scope of the present disclosure is not intended to be limited to the particular embodiments of the process, machine, manufacture, and composition of matter, means, methods, steps, and operations described in the specification. As those skilled in the art will readily appreciate from the disclosure of the present disclosure, processes, machines, manufacture, composition of matter, means, methods, steps, or operations presently existing or later to be developed that perform substantially the same function or achieve substantially the same result as the corresponding embodiments described herein may be utilized according to the present disclosure. Accordingly, the appended claims are intended to include within their scope processes, machines, manufacture, and compositions of matter, means, methods, steps, or operations. In addition, each claim constitutes a separate embodiment, and the combination of various claims and embodiments are within the scope of the disclosure.

The methods, processes, or operations according to embodiments of the present disclosure can also be implemented on a programmed processor. However, the controllers, flowcharts, and modules may also be implemented on a general purpose or special purpose computer, a programmed microprocessor or microcontroller and peripheral integrated circuit elements, an integrated circuit, a hardware electronic or logic circuit such as a discrete element circuit, a programmable logic device, or the like. In general, any device on which resides a finite state machine capable of implementing the flowcharts shown in the figures may be used to implement the processor functions of the present disclosure.

An alternative embodiment preferably implements the methods, processes, or operations according to embodiments of the present disclosure on a non-transitory, computer-readable storage medium storing computer programmable instructions. The instructions are preferably executed by computer-executable components preferably integrated with a network security system. The non-transitory, computer-readable storage medium may be stored on any suitable computer readable media such as RAMs, ROMs, flash memory, EEPROMs, optical storage devices (CD or DVD), hard drives, floppy drives, or any suitable device. The computer-executable component is preferably a processor, but the instructions may alternatively or additionally be executed by any suitable dedicated hardware device. For example, an embodiment of the present disclosure provides a non-transitory, computer-readable storage medium having computer programmable instructions stored therein.

While the present disclosure has been described with specific embodiments thereof, it is evident that many alternatives, modifications, and variations may be apparent to those skilled in the art. For example, various components of the embodiments may be interchanged, added, or substituted in the other embodiments. Also, all of the elements of each figure are not necessary for operation of the disclosed embodiments. For example, one of ordinary skill in the art of the disclosed embodiments would be able to make and use the teachings of the present disclosure by simply employing the elements of the independent claims. Accordingly, embodiments of the present disclosure as set forth herein are intended to be illustrative, not limiting. Various changes may be made without departing from the spirit and scope of the present disclosure.

Even though numerous characteristics and advantages of the present disclosure have been set forth in the foregoing description, together with details of the structure and function of the invention, the disclosure is illustrative only. Changes may be made to details, especially in matters of shape, size, and arrangement of parts, within the principles of the invention to the full extent indicated by the broad general meaning of the terms in which the appended claims are expressed.

What is claimed is:

1. A method of processing a low-dose computed tomography (CT) image, comprising:
receiving a first chest image, the first chest image generated by a low-dose CT method;
detecting at least one lung nodule in the first chest image;
determining at least one lung nodule region of the first chest image based on the at least one lung nodule; and
classifying the at least one lung nodule region based on a first set of radiomics features of the at least one lung nodule region of the first chest image to obtain a nodule score of the at least one lung nodule in the lung nodule region.

2. The method of claim 1, wherein detecting the at least one lung nodule comprises:
obtaining one or more sections of the first chest image;
detecting the at least one lung nodule in the first chest image based on the one or more sections of the first chest image.

3. The method of claim 2, wherein the one or more sections of the first chest image include sections along at least one of: a sagittal plane, a coronal plane, an axial plane, a first plane inclined 30 degrees from the coronal plane to the sagittal plane, a second plane inclined 30 degrees from the coronal plane to the axial plane, a third plane inclined 15 degrees from the sagittal plane to the coronal plane, or a fourth plane inclined 15 degrees from the sagittal plane to the axial plane.

4. The method of claim 1, wherein determining the at least one lung nodule region comprises:
obtaining a boundary of each of the at least one lung nodule region; and
calculating a size of each of the at least one lung nodule region based on the boundary of the corresponding lung nodule.

5. The method of claim 4, wherein classifying the at least one lung nodule region comprises:
determining a texture type of each of the at least one lung nodule region based on the first set of radiomics features;
determining a margin type of each of the at least one lung nodule in the lung nodule region based on the first set of radiomics features; and
determining the nodule score of the at least one lung nodule region based on the sizes, the texture types, the margin types of the at least one lung nodule region.

6. The method of claim 5, wherein the margin type includes sharp circumscribed, lobulated, indistinct, and speculated, the texture type includes solid, sub-solid, and ground glass opacity.

7. The method of claim 1, further comprising determining a location of the at least one lung nodule.

8. The method of claim 7, wherein the location of the at least one lung nodule includes a right upper lobe, a right middle lobe, a right lower lobe, a left upper lobe, a left lower lobe, and a lingular lobe.

9. The method of claim 1, wherein classifying the at least one lung nodule region is based on the first set of radiomics features and a first set of slice features of the at least one lung nodule region of the first chest image.

10. The method of claim 1, further comprising:
extracting a heart region in the first chest image by using a U-Net model;
determining a coronary artery calcification (CAC) score of the heart region by an transferred Efficient Net model.

11. The method of claim 10, further comprising providing a treatment recommendation based on the CAC score.

12. The method of claim 10, wherein the transferred Efficient Net model is trained from a pre-trained model for heart full-dose reference CT images and a low-dose reference CT image captured from a same region.

13. A device of processing a low-dose computed tomography (CT) image, comprising:
a processor; and
a memory coupled with the processor,
wherein the processor executes computer-readable instructions stored in the memory to perform operations, and the operations comprise:
receiving a first chest image, the first chest image generated by a low-dose CT method;
extracting a heart region in the first chest image by using a U-Net model; and
determining a coronary artery calcification (CAC) score of the heart region by an transferred Efficient Net model.

14. The device of claim 13, wherein the operations further comprises providing a treatment recommendation based on the CAC score.

15. The device of claim 13, wherein the transferred Efficient Net model is trained from a pre-trained model for heart full-dose reference CT images and a low-dose reference CT image captured from a same region.

16. The device of claim 13, wherein the operations further comprises:
detecting at least one lung nodule in the first chest image;
determining at least one lung nodule region of the first chest image based on the at least one lung nodule; and
classifying the at least one lung nodule region based on a first set of radiomics features of the at least one lung nodule region of the first chest image to obtain a nodule score of the at least one lung nodule in the lung nodule region.

17. The device of claim 16, wherein the operations further comprises:
obtaining a boundary of each of the at least one lung nodule region; and
calculating a size of each of the at least one lung nodule region based on the boundary of the corresponding lung nodule.

18. The device of claim 17, wherein the operations further comprises:
determining a texture type of each of the at least one lung nodule region based on the first set of radiomics features;
determining a margin type of each of the at least one lung nodule in the lung nodule region based on the first set of radiomics features; and
determining the nodule score of the at least one lung nodule region based on the sizes, the texture types, the margin types of the at least one lung nodule region.

19. The device of claim 18, wherein the margin type includes sharp circumscribed, lobulated, indistinct, and speculated, the texture type includes solid, sub-solid, and ground glass opacity.

20. The device of claim 16, wherein classifying the at least one lung nodule region is based on the first set of radiomics features and a first set of slice features of the at least one lung nodule region of the first chest image.
